(19) United States
(12) Patent Application Publication
(10) Pub. No.: US 2011/0218559 A1
(43) Pub. Date: Sep. 8, 2011

(54) OXIDIZED POLYPROPYLENE MESH MATERIALS FOR TISSUE IN GROWTH

(76) Inventor: Yelena G. Tropsha, Plymouth, MN (US)

(21) Appl. No.: 13/042,199

(22) Filed: Mar. 7, 2011

Related U.S. Application Data
(60) Provisional application No. 61/311,585, filed on Mar. 8, 2010.

(51) Int. Cl. A61B 17/00

(52) U.S. Cl. 606/151

(57) ABSTRACT

Oxidized polypropylene meshes are described that help promote tissue in growth wherein the oxidized surface attracts macrophages and helps reduce inflammation about the area to which it is implanted.

### CROSS REFERENCE TO RELATED APPLICATIONS

This application claims benefit of U.S. Provisional Patent Application Ser. No. 61/311,585 filed on Mar. 8, 2010, entitled “Oxidized Polypropylene Mesh Materials for Tissue In Growth,” the contents of which are incorporated in their entirety herein by reference.

### FIELD

Aspects generally relate to surface modified propylene mesh materials suitable for tissue in growth, in particular, for use in pelvic floor reconstruction and/or support. Aspects generally relate to meshes for use in supporting tissues, organs, parts of organs, or other such anatomical structures. The meshes may be used in a variety of pelvic floor reconstruction or stabilization procedures, including treatment of stress urinary incontinence, pelvic organ prolapse (i.e., “POP”) conditions such as cystoceles, rectoceles, enteroceles, or enterocystoceles. More particularly, the present invention relates to prefabricated meshes, methods of making the meshes, and kits including the patches.

### BACKGROUND

Damage to the pelvic floor is a serious medical condition which may occur during delivery of an infant or due to injury to the vesicovaginal fascia. These herniations are serious medical problems that can severely and negatively impact a patient both physiologically and psychologically.

Treatment of these conditions requires repositioning of the protruding organs or portions thereof. Existing tissue is often compromised facilitating the need to use a synthetic patch. Current medical procedures for repositioning the protruding organs or portions thereof may be time consuming or invasive. Hence, it is desirable to reduce the amount of time which these procedures require and the invasiveness of the procedures.

For example, an implantable support, such as a sheet or patch of flexible material, is used to provide support to weakened or destabilized tissue of a patient. The implantable support is used to treat a variety of conditions, including, for example, closing a hernia and providing suburethral stabilization.

Some materials currently being used to manufacture such supports fail to attach adequately to surrounding tissue or experience undesirable deformation after implantation. Such conditions often require an additional surgical procedure and/or result in discomfort to the patient.

In one particular procedure, commonly known as a transvaginal or pubovaginal sling procedure, a patch or strip of biological tissue is used to provide suburethral stabilization for female patients experiencing bladder dysfunction, such as stress urinary incontinence. However, ends of the strip are friable and tend to weaken or rupture upon penetration by a relatively large needle.

Therefore, a need exists for an approach that overcomes one or more of the current disadvantages noted above.

### BRIEF SUMMARY

The present invention provides a surface oxidized propylene mesh which will surprisingly facilitate tissue in growth, methods of use thereof, kits that include the oxidized mesh and methods to prepare the surface oxidized mesh.

In one aspect, the pore size of the surface oxidized propylene mesh is at least about 75 μm to about 1500 μm, more particularly from about 100 μm to about 1200 μm, even more particularly from about 150 μm to about 750 μm and also from about 75 μm to about 750 μm and all values and ranges therebetween.

In still another aspect, the surface oxidized propylene mesh is shelf-stable after an oxidizing treatment for at least 3 months to at least 6 months, more particularly from about 1 month to about 6 months, from about 2 months to about 5 months and from about 3 months to about 4 months at ambient conditions. In one specific embodiment, the oxidized polypropylene mesh can be stored at ambient conditions in a sealed container or bag with nitrogen or argon atmosphere.

Surface oxidation of the propylene mesh can be effected by plasma oxidation, UV/air oxidation, corona treatment, or electron beam with an oxidant. Suitable oxidants include but are not limited to, for example, moist air, ionized air, oxygen, NO₂ or CO₂.

The surface oxidized propylene meshes described herein can be used as pelvic slings. The mesh materials can be used to prevent or reduce a pelvic organ prolapse condition comprising by providing a surface oxidized mesh support member and securing the support member to sacrospinous ligaments or transobtuator. Typically, a transvaginal or pubovaginal sling implantation procedure can be utilized. In one embodiment, a portion or the surface oxidized mesh is secured about the neck of the bladder.

The unique surface characteristics of the oxidized mesh are advantageous for tissue in growth. In one embodiment, the oxidized surface of the polypropylene mesh is provided with a lower surface energy having an increased hydrophilicity such that the mesh material exhibits a water contact angle of about 40 degrees. The oxidized surface of the polypropylene mesh helps to improve protein absorption at the surface such that cellular adhesion occurs. The oxidized surface of the polypropylene mesh helps to promote migration of macrophages, fibroblasts, VEGF neutrophils, monocytes, leukocytes, lymphocytes, kininogen and/or fibrinogen such that tissue migrates into and about the mesh while stunting inflammatory reactions and/or retarding platelet deposition. This in turn helps to facilitate growth of myoblasts about the mesh and tissue such that promotion of muscular tissue growth occurs. This tissue in growth helps to secure the support in place such that the patient does not need to be concerned that the support may lose the ability to support the area that requires support, e.g., urethra, vagina, uterus, intestine, etc.

Additional advantages of the oxidized surface of the polypropylene mesh include reduced infection rates in comparison to meshes that do not include oxidized surfaces and/or increased biocompatibility of polypropylene meshes that do not have an oxidized surface.

Suitable polypropylene meshes that are treatable with the surface oxidation processes described herein includes those that are commercially available from Coloplast Corporation, Minneapolis, USA, such as mesh products sold under the trademarks ARIS, NOVASILK, VIRTUE and EXAIR.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### DETAILED DESCRIPTION

In the specification and in the claims, the terms “including” and “comprising” are open-ended terms and should be interpreted to mean “including, but not limited to. . . . ” These terms encompass the more restrictive terms “consisting essentially of” and “consisting of.”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

Damage to the pelvic floor can result in a herniation of the bladder called a cystocele. Other similar conditions are known as rectoceles, enteroceles, enterocystoceles and urethrocele (prolapse of the urethra). A rectocele is a herniation of the rectum. An enterocele is formed when the intestine protrudes through a defect in the rectovaginal or vesicovaginal pouch and an enterocystocele is a double hernia in which both the bladder and the intestine protrude. These herniations are serious medical problems that can severely and negatively impact a patient both physiologically and psychologically.

Urinary incontinence is another conditions that can negatively impact the quality of an individual's well being. Such condition can range in severity from partial to complete loss of bladder control and patients afflicted with urinary incontinence can experience varying degrees of urine loss. In addition, it is known that urinary incontinence may change over time and that men and women with light incontinence, for example, may experience minimal leakage during the occurrence of a provocative event, such as laughing or coughing (stress incontinence), whereas men and women with heavy incontinence may experience continuous urine leakage.

Generally, urinary incontinence is not considered a disease, but rather a symptom or side effect of another medical condition. Some conditions known to cause male urinary incontinence include prostate surgery, and in particular total prostatectomy, head and spinal cord injury, infection, and certain diseases, such as cancer, Parkinson's disease and multiple sclerosis. Indeed, male incontinence can be caused simply by the aging process or emotional distress.

Female incontinence may be caused by weakened and (or) stretched pelvic muscles, which is associated with child-birth, pregnancy, trauma, prior surgical procedures, and estrogen loss.

Each case of incontinence, however, is unique and no two people are affected by incontinence in the same way. There are, however, well-recognized types of incontinence and various ways to treat the same. Stress incontinence, which is a common type of incontinence, may be characterized as urine leakage during a provocative event such as sneezing, laughing, lifting heavy objects, or when the patient engages in any type of exercise that puts pressure on the bladder. Urge incontinence occurs when the patient wants to urinate but is incapable of exercising restraint until reaching a restroom. Additional types of incontinence include: overflow incontinence, which occurs when the quantity of urine exceeds the capacity of the patient's bladder, and functional incontinence, which occurs when the patient has knowledge of the need to urinate but simply cannot access a restroom quickly enough due to a physical obstruction or debilitation.

There is also a condition referred to as intrinsic sphincter deficiency. Intrinsic sphincter deficiency describes a condition where the sphincter muscle (responsible for preventing urine being expelled from the bladder) has become weakened or atrophied to the point where it can no longer retain urine under normal circumstances.

The oxidized polypropylene materials described herein can be used in a variety of pelvic floor reconstruction or stabilization procedures, including treatment of (stress or sphincter) urinary incontinence, pelvic organ prolapse (POP) conditions such as cystoceles, rectoceles, enteroceles, enterocystoceles, or urethrocele all referred to generally as “POP” herein.

Suitable examples of implanting and securing mesh materials include those described in WO 2007/066169, U.S. Pat. Nos. 7,431,690, 7,559,885, 7,588,598, 6,197,036, 6,355,065, 6,599,318, US Publications 2006/0130848, 2004/0231678, or combinations thereof, the contents of which are incorporated herein by reference in their entirety for all purposes.

Essentially, the oxidized polypropylene mesh is inserted about the defective physiological area and secured such that the defective area is supported. After the positioning and insertion of the oxidized polypropylene mesh is accomplished, tissue in growth will occur and is stimulated by the oxidized surface. Since the polypropylene has been oxidized, tissue in growth occurs through the mesh such that the newly formed tissue integrates itself throughout the mesh and to itself. Therefore, the oxidized mesh acts as a support as well as a platform for the tissue to grow through and about the porous structure. Ultimately, the support provided by the polypropylene support mesh material can be inconsequential as the body has regenerated tissue that supports the individual's physiology.

Suitable methods to oxidize the polypropylene mesh include plasma oxidation, UV/air oxidation, corona treatment, or electron beam with an oxidant. Suitable oxidants include but are not limited to, for example, oxygen, ionized air, NO₂ or CO₂.

Oxidation of the surface is accomplished with corona discharge oxidation, plasma oxidation, electron beam (also referred to as electron curtain), or UV light exposure. The polypropylene mesh would be treated under oxidizing conditions with one or more of the processes to effect oxidation of the surface. Generally treatment times will range from a few seconds to up to 15 minutes. Typically the mesh is placed between two electrodes or in a chamber and subjected to the ionized/reactive gas. The charged particles then react with the surface of the polypropylene to impart an oxidized surface to a depth into the material from a monomolecular layer at the surface down into the material approximately 1-3 micrometers. The oxidized surface remains stable at ambient conditions for at least 3 months, more particularly about 6 months. Polypropylene materials with a high degree of crystallinity can provide stability for up to 1 year.

The phrase “stable” is intended to mean that the oxidized polypropylene mesh materials do not appreciably change in physical characteristics over a given period of time. That is, the ability to promote tissue in growth is not effected if the material is left at ambient conditions for a period of time prior to use, e.g., for at least about 3 month, more particularly about 4 to about 6 months. The surface treatment to the polypropylene mesh does not lose its ability to promote tissue in growth over such a period of time, and as such, the physical make up of the oxidized polypropylene mesh does not appreciably change to an extent that tissue in growth would not be improved over a native untreated sample of polypropylene mesh.

Generally, the process noted above, involve a gas cloud that has been excited by the application of energy. A cloud of fast moving particles is produced, including electrons, ions, atoms, free radicals, molecules and other metastable species. This energetic cloud is capable of reacting with the polypropylene surface in a variety of ways. Specific examples of these processes include corona discharge and plasma treatment. These processes may occur in a variety of gaseous environments such as air, or inert gas mixtures.

Corona discharge is produced by capacitative exchange of a gaseous medium which is present between two spaced electrodes, at least one of which is insulated from the gaseous medium by a dielectric barrier. Corona discharge is somewhat limited in origin to alternating currents because of its capacitative nature. It is a high voltage, low current phenomenon with voltages being typically measured in kilovolts and currents being typically measured in milliamperes. Corona discharges may be maintained over wide ranges of pressure and frequency. Pressures of from 0.2 to 10 atmospheres generally define the limits of corona discharge operation and atmospheric pressures generally are preferred. Frequencies ranging from 20 Hz to 100 MHz can conveniently be used: in particular ranges are from 500 Hz, especially 3000 Hz to 10 MHz.

When dielectric barriers are employed to insulate each of two spaced electrodes from the gaseous medium, the corona discharge phenomenon is frequently termed an electrodeless discharge, whereas when a single dielectric barrier is employed to insulate only one of the electrodes from the gaseous medium, the resulting corona discharge is frequently termed a semi-corona discharge. The term “corona discharge” is used throughout this specification to denote both types of corona discharge, i.e. both electrodeless discharge and semi-corona discharge.

Electron beam or electron curtain refers to a process by which electrons are emitted from a electron source (a cathode) that radiates out due to repeated scattering of the electrons caused by the electrons repeatedly colliding with inert molecules in a processing space inside a vacuum chamber.

In another aspect, a method to oxidize the polypropylene mesh is with UV light. For example, a surgeon can subject the polypropylene mesh to UV treatment just prior to affixing the oxidized polypropylene mesh into the patient. Typical UV treatment of the polypropylene mesh is from a few seconds, a few minutes to a few hours prior to insertion. Suitable UV light sources can be provided in an operatory environment so that the surgeon can oxidize the mesh just prior to the procedure. Acceptable UV light sources are available from industrial sources such as DYMAX, Fusion UV Systems, Inc., Wedeco UV Lamp Manufactueres, for example. The UV light source can be a UV flood lamp, UV focused beam lamp, UV conveyor system, UV cabinet, UV hand lamp, etc.

Optionally, the polypropylene can be treated with a radical generator such as a photoinitiator to help oxidize the polypropylene surfaced without comprising the bulk properties of the polypropylene. Suitable photoinitiators include but are not limited to azobisisobutyronitrile (AIBN), benzoyl peroxide (BPO), and combinations thereof. The polypropylene mesh can be dipped into a solution of the photoinitiator, removed from the solution and then treated with UV light. The concentration of the photoinitiator can be varied from 1% to about 10% by weight and is not limiting. Suitable solvents for the photoinitiator are water or aqueous alcoholic solutions (ethanol, propyl alcohol, isopropyl alcohol). After the polypropylene mesh has undergone UV treatment, it is rinsed with water to remove any remaining photoinitiator prior to use within the patient.

The following paragraphs enumerated consecutively from 1 through 26 provide for various aspects of the present invention. In one embodiment, in a first paragraph (1), the present invention provides a surface oxidized propylene mesh which will facilitate tissue in growth.
2. The surface oxidized propylene mesh of paragraph 1, wherein the pore size of the mesh is at least about 75 μm to about 1500 μm.
3. The surface oxidized propylene mesh of either of paragraphs 1 or 2, wherein the oxidized propylene mesh has a water contact angle of about 40 degrees.
4. The surface oxidized propylene mesh of any of paragraphs 1 through 3, wherein the mesh is stable for at least 3 months.
5. The surface oxidized propylene mesh of paragraph 4, wherein the mesh is stable for at least about 6 months.
6. The surface oxidized propylene mesh of any of paragraphs 1 through 5, wherein the surface oxidation is effected by plasma oxidation, UV/air oxidation, corona treatment, or electron beam with an oxidant.
7. The surface oxidized propylene mesh of paragraph 6, wherein the oxidant is ionized air, NO₂ or CO₂.
8. A method to promote tissue in growth about a polypropylene support comprising the step of securing an oxidized polypropylene mesh about a site requiring support.
9. The method of paragraph 8, wherein the step of securing the oxidized polypropylene mesh is by a trans-obturator technique.
10. The method of either of paragraphs 8 or 9, wherein the site is about the urethra.
11. The method of either of paragraphs 8 or 9, wherein the site is about the uterus.
12. The method of any of paragraphs 8 through 11, wherein the pore size of the mesh is at least about 75 μm to about 1500 μm.
13. The method of any of paragraphs 8 through 12, wherein the oxidized propylene mesh has a water contact angle of about 0 and 30 degrees.
14. The method of any of paragraphs 8 through 13, wherein the mesh is stable for at least 3 months.
15. The method of paragraph 14, wherein the mesh is stable for at least about 6 months.
16. The of any of paragraphs 8 through 15, wherein the surface oxidation is effected by plasma oxidation, UV/air oxidation, corona treatment, or electron beam with an oxidant.
17. The method of paragraph 16, wherein the oxidant is ionized air, NO₂ or CO₂.
18. A method to modify the surface of a polypropylene mesh comprising the step of subjecting a polypropylene mesh to an oxidizing environment, wherein oxidation of the surface occurs by plasma oxidation, UV/air oxidation, corona treatment, or electron beam with an oxidant.
19. The method of paragraph 18, wherein the oxidant is ionized air, NO₂ or CO₂.
20. The method of any of paragraphs 19 through 19, wherein the pore size of the mesh is at least about 75 μm to about 1500 μm.
21. The method of any of paragraphs 18 through 20, wherein the oxidized propylene mesh has a water contact angle of about 40 degrees.
22. The method of any of paragraphs 19 through 24, wherein the mesh is stable for at least 3 months.
26. The method of paragraph 25, wherein the mesh is stable for at least about 6 months.

The invention will be further described with reference to the following non-limiting Examples. It will be apparent to those skilled in the art that many changes can be made in the embodiments described without departing from the scope of the present invention. Thus the scope of the present invention should not be limited to the embodiments described in this application, but only by embodiments described by the language of the claims and the equivalents of those embodiments. Unless otherwise indicated, all percentages are by weight.

### Examples

A polypropylene mesh would be treated under plasma process conditions. For example, a vacuum chamber contains two conducting electrodes which would be placed opposite each other in the chamber. One electrode would be connected to an RF power supply and the other electrode would be connected to a ground. Alternatively, a DC ion source may be used for ignition of the plasma. The mesh would be placed in contact with the ground electrode.

The vacuum chamber would be connected to a source of gasified liquid or gas that could include air, oxygen, or mixtures thereof. The connections to the gases are typically through mass flow meters. In one configuration, the RF-driven electrode would be a shower head electrode, used for the injection of the process gas. The shower head concept would lead to a very good uniformity of gas injection on the whole surface.

After a base chamber pressure would be reached, a first gas such as hydrogen can be introduced, followed by a second gas (or combination of gases) into the chamber in a various ratios. It is also possible to use argon, oxygen, ammonia (NH₃), or helium as the pretreatment gas. Mixtures of one or more of these gases are within the scope of the present invention.

The plasma can be ignited by the RF power supply producing about a 40 KHz to about a 2.45 GHz frequency. Alternatively, a DC ion source may be used to ignite the plasma. The power would be between about 0.1 to about 1 W/cm², of forward power and the mesh surface is exposed to the plasma for about 120 seconds, preferably exposure is for approximately 60 seconds. Thus the speed of mesh feed would be about 25 feet/minute. The reaction would be conducted at room temperature.

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A surface oxidized propylene mesh which will facilitate tissue in growth.

2. The surface oxidized propylene mesh of claim 1, wherein the pore size of the mesh is at least about 75 μm to about 1500 μm.

3. The surface oxidized propylene mesh of claim 1, wherein the oxidized propylene mesh has a water contact angle of about 0 to about 30 degrees.

4. The surface oxidized propylene mesh of claim 1, wherein the mesh is stable for at least 3 months.

5. The surface oxidized propylene mesh of claim 4, wherein the mesh is stable for at least about 6 months.

6. The surface oxidized propylene mesh of claim 1, wherein the surface oxidation is effected by plasma oxidation, UV/air oxidation, corona treatment, or electron beam with an oxidant.

7. The surface oxidized propylene mesh of claim 6, wherein the oxidant is ionized air, oxygen, NO₂ or CO₂.

8. A method to promote tissue in growth about a polypropylene support comprising the step of securing an oxidized polypropylene mesh about a site requiring support.

9. The method of claim 8, wherein the step of securing the oxidized polypropylene mesh is by a trans-obturator technique.

10. The method of claim 8, wherein the site is about the urethra.

11. The method of claim 8, wherein the site is about the uterus.

12. The method of claim 8, wherein the pore size of the mesh is at least about 75 μm to about 1500 μm.

13. The method of claim 8, wherein the oxidized propylene mesh has a water contact angle of about 0 to about 30 degrees.

14. The method of claim 8, wherein the mesh is stable for at least 3 months.

15. The method of claim 14, wherein the mesh is stable for at least about 6 months.

16. The of any of claim 8, wherein the surface oxidation is effected by plasma oxidation, UV/air oxidation, corona treatment, electron beam with an oxidant.

17. The method of claim 16, wherein the oxidant is ionized air, oxygen, NO₂ or CO₂.

18. A method to modify the surface of a polypropylene mesh comprising the step of subjecting a polypropylene mesh to an oxidizing environment, wherein oxidation of the surface occurs by plasma oxidation, UV/air oxidation, corona treatment, or electron beam with an oxidant.

19. The method of claim 18, wherein the oxidant is ionized air, NO₂, oxygen or CO₂.

20. The method of claim 18, wherein the pore size of the mesh is at least about 75 μm to about 1500 μm.

21. The method of claim 18, wherein the oxidized propylene mesh has a water contact angle of about 0 to about 30 degrees.

22. The method of claim 18, wherein the mesh is stable for at least 3 months.

23. The method of claim 18, wherein the mesh is stable for at least about 6 months.
